(19) United States
(12) Patent Application Publication
(10) Pub. No.: US 2018/0033965 A1
(43) Pub. Date: Feb. 1, 2018

(54) NOVEL DIAMINE COMPOUND AND ORGANIC ELECTROLUMINESCENT DEVICE USING THE SAME

(71) Applicant: E-RAY OPTOELECTRONICS TECHNOLOGY CO., LTD., Taoyuan City (TW)

(72) Inventors: Heh-Lung Huang, Taoyuan City (TW); Po-Wei Hsu, Taoyuan City (TW); Cheng-Chung Yao, Taoyuan City (TW); Teng-Chih Chao, Taoyuan City (TW)

(21) Appl. No.: 15/379,539

(22) Filed: Dec. 15, 2016

(30) Foreign Application Priority Data
Jul. 26, 2016 (TW) 105123561

(51) Int. Cl. H01L 51/00; C07D 333/76; C07C 211/61; C07C 211/58; C09K 11/06; C09K 11/02

(52) U.S. Cl. CPC H01L 51/006; C09K 11/06; C09K 11/02; C07C 211/61; C07C 211/58; C07D 333/76; H01L 51/0061; H01L 51/5221

(57) ABSTRACT

Provided is a compound represented by formula (I),

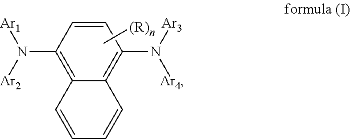

wherein R represents substituted or unsubstituted (C1-C10)alkyl; each of Ar₁, Ar₂, Ar₃ and Ar₄ independently represents substituted or unsubstituted (C6-C30)aryl, or substituted or unsubstituted (5 to 30-membered)heteroaryl; and n represents an integer of from 0 to 4. Also provided is an organic electroluminescent device including the compound of formula (I).

### TECHNICAL FIELD

The present disclosure relates to a novel diamine compound and an organic electroluminescent element using the same.

### BACKGROUND

Recently, organic electroluminescent elements (OLED) have drawn attention due to their properties of self-emitting, low driving voltage, high efficiency, high luminance, light weight, thinness, and broad color gamut. Development of novel organic materials has gained more attention, in order to satisfy the applications of organic electroluminescent elements.

Most amine compounds, which have been mainly used in optical conductors in xerography, have a very high hole mobility of about 10⁻³ cm/Vs, such that they can be used as hole transport materials, host materials or fluorescent materials in organic electroluminescent elements.

The use of conventional blue fluorescent materials, such as simple small organic molecules, for example, derivatives of anthracene, carbazole, and coumarin, in organic electroluminescent elements has been known broadly. However, problems, such as photochromic impurity, poor luminous efficiency, and short operational life are commonly faced by the conventional fluorescent materials used in organic electroluminescent elements.

Therefore, there is continuous need for novel luminescent materials, particularly fluorescent materials emitting blue light.

SUMMARY

The present disclosure provides a novel diamine compound and an organic electroluminescent element using the same. The organic electroluminescent element emits light with the color from dark blue to blue and has a relative longer operational life and a low driving voltage.

The diamine compound of the present disclosure is shown in formula (I):

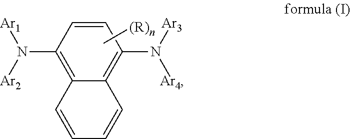

wherein R represents substituted or unsubstituted (C1-C10)alkyl;

each of Ar₁, Ar₂, Ar₃ and Ar₄ independently represents substituted or unsubstituted (C6-C30)aryl, or substituted or unsubstituted (5 to 30-membered)heteroaryl; and

n represents an integer of from 0 to 4.

The compound of formula (I) of the present disclosure can also be represented by formula (II):

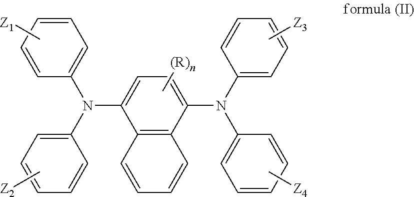

wherein R represents substituted or unsubstituted (C1-C10)alkyl;

each of Z₁, Z₂, Z₃ and Z₄ independently represents substituted or unsubstituted (C1-C10)alkyl, substituted or unsubstituted (C3-C10)cycloalkyl, substituted or unsubstituted (C6-C30)aryl, or substituted or unsubstituted (5 to 30-membered)heteroaryl; or at least one of Z₁, Z₂, Z₃ and Z₄ is fused with the adjacent benzene ring of formula (II) to form substituted or unsubstituted (C9-C30)aryl; or at least one of Z₁, Z₂, Z₃ and Z₄ is fused with the adjacent benzene ring of formula (II) to form substituted or unsubstituted (10 to 30-membered)heteroaryl.

The present disclosure further provides an organic electroluminescent element comprising: a cathode; an anode; and an organic layer formed between the cathode and the anode, and the organic layer comprises the compound of formula (I) described above.

The organic layer of the organic electroluminescent element of the present disclosure can be an electron transport layer, an electron injection layer, a light emitting layer, a hole blocking layer, or an electron blocking layer. In addition to the organic layer, the organic electroluminescent element can further include at least one layer, which differs from the organic layer and is selected from the group consisting of an electron transport layer, an electron injection layer, a light emitting layer, a hole blocking layer, and an electron blocking layer.

### BRIEF DESCRIPTION OF THE DRAWINGS

FIG. 1 is a schematic sectional view of an organic electroluminescent element of one example of the present disclosure;

FIG. 2 is a schematic sectional view of an organic electroluminescent element of another example of the present disclosure;

FIG. 3 is a schematic sectional view of an organic electroluminescent element of still another example of the present disclosure;

FIG. 4 is the electroluminescence spectra of the organic electroluminescent elements; and

FIG. 5 is the electroluminescence spectra of the organic electroluminescent elements.

### DETAILED DESCRIPTION OF THE EMBODIMENTS

The present disclosure will be explained in details by the following preferred examples to allow one having ordinary skill in the art to readily conceive benefits and effects disclosed by the present specification.

Ranges cited and values disclosed herein are all inclusive and combinable. For example, when any integer or a point value falls within the range cited herein, the point value or number can be used as the lower limit or the upper limit to derive a sub-range. Additionally, all of the groups listed herein, such as the groups or substituents with reference to R, Ar₁, Ar₂, Ar₃, Ar₄, Z₁, Z₂, Z₃, and Z₄, are all combination with other groups in formula (I) or formula (II).

According to the present disclosure, the diamine compound capable of being applied in an OLED is shown in formula (I):

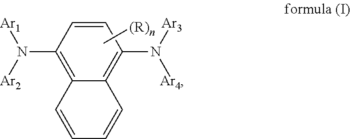

wherein R represents substituted or unsubstituted (C1-C10)alkyl;

each of Ar₁, Ar₂, Ar₃, and Ar₄ independently represents substituted or unsubstituted (C6-C30)aryl, or substituted or unsubstituted (5 to 30-membered)heteroaryl and

n represents an integer of 0 to 4.

In one embodiment, Ar₁ and Ar₃ are the same, and Ar₂ and Ar₄ are the same.

In one embodiment, R represents (C1-C10)alkyl.

In one embodiment, n represents 0, 1, or 2.

In one embodiment, R represents (C1-C10)alkyl; each of Ar₁, Ar₂, Ar₃, and Ar₄ independently represents substituted (C6-C30)aryl, wherein the substituent of the substituted (C6-C30)aryl is at least one selected from the group consisting of (C1-C10)alkyl, (C1-C1 0)alkyl substituted with (C6-C30)aryl, (C3-C1 0)cycloalkyl, and (5 to 30-membered)heteroaryl; and n represents 0, 1, or 2.

In one embodiment, at least one of Ar₁, Ar₂, Ar₃ and Ar₄ is (C6-C30)aryl substituted with (C1-C10)alkyl or (C3-C10)cycloalkyl. Additionally, in the diamine compound of formula (I), each of Ar₁, Ar₂, Ar₃ and Ar₄ can be (C6-C30)aryl substituted with (C1-C10)alkyl or (C3-C10)cycloalkyl.

In one embodiment, at least one of Ar₁, Ar₂, Ar₃ and Ar₄ is (C6-C30)aryl substituted with (C2-C10)alkyl or (C3-C10)cycloalkyl. Additionally, in the diamine compound of formula (I), each of Ar₁, Ar₂, Ar₃ and Ar₄ can be (C6-C30)aryl substituted with (C2-C10)alkyl or (C3-C10)cycloalkyl (for example, substituted with propyl or cyclohexyl).

In one embodiment, n is 0, and at least one of Ar₁, Ar₂, Ar₃ and Ar₄ is (C6-C30)aryl substituted with (C3-C10)alkyl or (C3-C10)cycloalkyl. Additionally, in the diamine compound of formula (I), each of Ar₁, Ar₂, Ar₃ and Ar₄ can be (C6-C30)aryl substituted with (C3-C10)alkyl or (C3-C10)cycloalkyl (e.g., substituted with propyl or cyclohexyl).

In one embodiment, each of Ar₁, Ar₂, Ar₃ and Ar₄ independently represents 9,9-dimethylfluorenyl, biphenyl, propylphenyl, cyclohexylphenyl, methylphenyl, phenyl, dibenzothienylphenyl, or phenylpropylphenyl.

In one embodiment, each of Ar₁, Ar₂, Ar₃ and Ar₄ independently represents

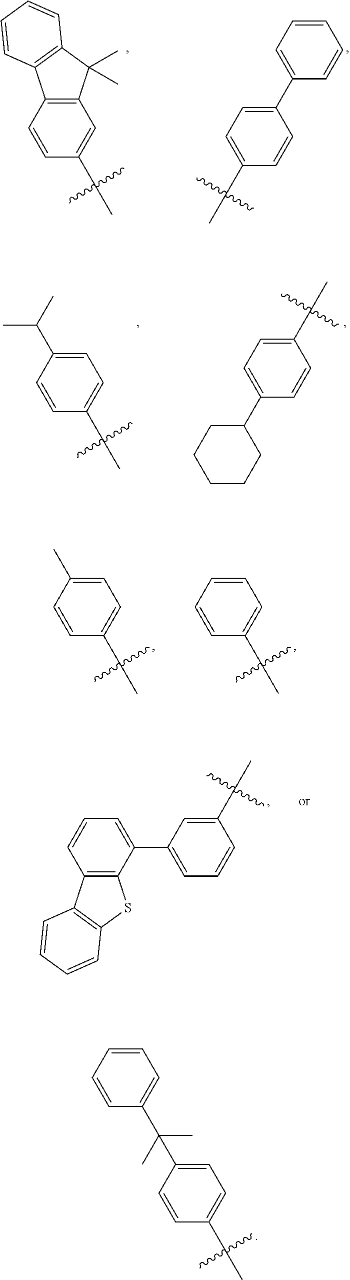

The compound of formula (I) of the present disclosure can be shown in formula (II):

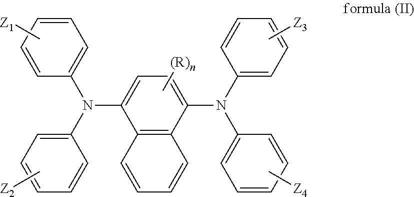

wherein R represents substituted or unsubstituted (C1-C10)alkyl;

each of Z₁, Z₂, Z₃ and Z₄ independently represents substituted or unsubstituted (C1-C10)alkyl, substituted or unsubstituted (C3-C10)cycloalkyl, substituted or unsubstituted (C6-C30)aryl, or substituted or unsubstituted (5 to 30-membered)heteroaryl; or at least one of Z₁, Z₂, Z₃ and Z₄ is fused with the adjacent benzene ring of formula (II) to form substituted or unsubstituted (C9-C30)aryl; or at least one of Z₁, Z₂, Z₃ and Z₄ is fused with the adjacent benzene ring of formula (II) to form substituted or unsubstituted (10 to 30-membered)heteroaryl.

In one embodiment, each of Z₁, Z₂, Z₃ and Z₄ independently represents methyl, isopropyl, phenylisopropyl, cyclohexyl, dibenzothienyl, or at least one of Z₁, Z₂, Z₃ and Z₄ is fused with the adjacent benzene ring of formula (II) to form 9,9-dimethylfluorenyl.

In one embodiment, at least one of Z₁, Z₂, Z₃, and Z₄ is substituted with at least one selected from the group consisting of (C-C10)alkyl, (C3-C10)cycloalkyl, (C6-C30)aryl and (5 to 30-membered)heteroaryl.

Herein, expression of “substituted” in “substituted or unsubstituted” means that a hydrogen atom in a given functional group is replaced with another atom or a group (i.e., a substituent). The substituents are each independently at least one selected from the group consisting of deuterium; halogen; (C1-C30)alkyl; (C1-C30)alkoxy; (C6-C30)aryl; (5 to 30-membered)heteroaryl, wherein the (5 to 30-membered)heteroaryl can be substituted with (C6-C30)aryl; (5 to 30-membered)heteroaryl substituted with (C6-C30)aryl; (C3-C30)cycloalkyl; (5 to 7-membered)heterocycloalkyl; tri(C1-C30)alkylsilyl; tri(C1-C30)arylsilyl; di(C1-C30)alkyl(C6-C30)arylsilyl; (C1-C30)alkyldi(C6-C30)arylsilyl; (C2-C30)alkenyl; (C2-C30)alkynyl; cyano; di(C1-C30)alkylamino; di(C6-C30)arylamino; (C1-C30)alkyl(C6-C30)arylamino; di(C6-C30)arylboryl; di(C1-C30)alkylboryl; (C1-C30)alkyl(C6-C30)arylboryl; (C6-C30)aryl(C1-C30)alkyl; (C1-C30)alkyl(C6-C30)aryl; carboxyl; nitro; and hydroxyl.

The term “alkyl” used herein includes a linear alkyl group, such as, methyl, ethyl, n-propyl, isopropyl, n-butyl, isobutyl, and tert-butyl.

The term “cycloalkyl” used herein includes a cycloalkyl group, such as, cyclopropyl, cyclobutyl, cyclopentyl, and cyclohexyl.

The term “aryl” used herein refers to a monocyclic ring or a fused ring derived from an aromatic hydrocarbon, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthryl, phenylphenanthryl, anthracyl, indenyl, triphenylenyl, pyrenyl, naphthacenyl, perylenyl, chrysenyl, napthacenyl, fluoranthenyl , acenaphthyl, and the like.

The term “(5 to 30-membered)heteroaryl” used herein refers to an aryl group having 5 to 30 ring atoms containing at least one heteroatom selected from the group consisting of B, N, O, S, P(═O), Si, and P; can be a monocyclic ring or a fused ring formed by combining with at least one benzene ring; is partially saturated; is formed by connecting at least one heteroaryl or aryl to a heteroaryl through one or more single bond(s); and includes a monocyclic ring-type heteroaryl, such as, furyl, thienyl, pyrryl, imidazolyl, pyrazolyl, thiazolyl, thiadizolyl, isothiazolyl, isoxazolyl, oxazolyl, oxadiazolyl, triazinyl, tetrazinyl, triazolyl, tetrazolyl, furazanyl, pyridyl, pyrazinyl, pyrimidyl, and pyridazinyl, and a fused ring-type heteroaryl, such as, benzofuranyl, benzothienyl, isobenzofuranyl, dibenzofuranyl, dibenzothienyl, benzimidazolyl, benzothiazolyl, benzoisothiazolyl, benzoisoxazolyl, benzoxazolyl, isoindolyl, indolyl, indazolyl, benzothiadizolyl, quinolyl, isoquinolyl, cinnolinyl, quinazolyl, quinoxalyl, carbazolyl, phenoxazinyl, phenanthridinyl, benzodioxolyl, and acridanyl.

Examples of aforementioned compound of formula (I) are compounds A1 to A13 listed in Table 1 below, but they are not limited thereto.

TABLE 1

| | |
|---|---|
| 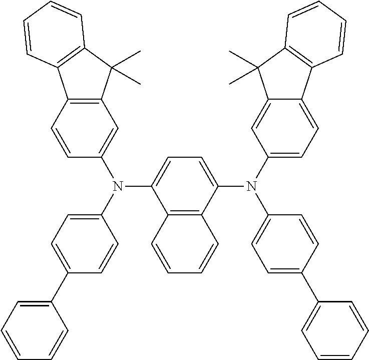 [SMILES: CC1(C)c2ccccc2-c2ccc(N(c3ccc(-c4ccccc4)cc3)c3ccc(N(c4ccc(-c5ccccc5)cc4)c4ccc5c(c4)C(C)(C)c4ccccc4-5)c4ccccc34)cc21] | A1 |
| 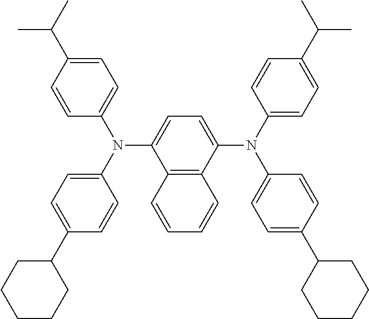 [SMILES: CC(C)c1ccc(N(c2ccc(C3CCCCC3)cc2)c2ccc(N(c3ccc(C(C)C)cc3)c3ccc(C4CCCCC4)cc3)c3ccccc23)cc1] | A2 |
| 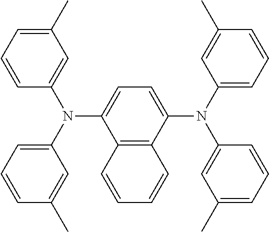 [SMILES: Cc1cccc(N(c2cccc(C)c2)c2ccc(N(c3cccc(C)c3)c3cccc(C)c3)c3ccccc23)c1] | A3 |
| 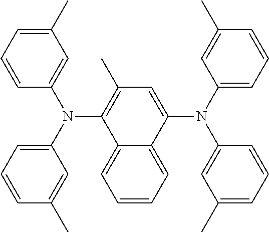 [SMILES: Cc1cccc(N(c2cccc(C)c2)c2cc(C)c(N(c3cccc(C)c3)c3cccc(C)c3)c3ccccc23)c1] | A4 |
| 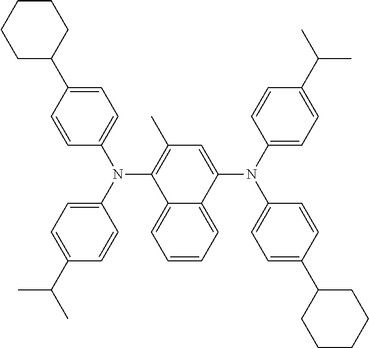 [SMILES: Cc1cc(N(c2ccc(C(C)C)cc2)c2ccc(C3CCCCC3)cc2)c2ccccc2c1N(c1ccc(C(C)C)cc1)c1ccc(C2CCCCC2)cc1] | A5 |
| 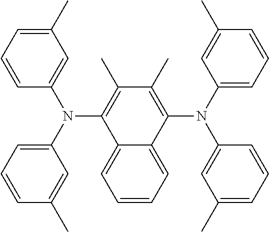 [SMILES: Cc1cccc(N(c2cccc(C)c2)c2c(C)c(C)c(N(c3cccc(C)c3)c3cccc(C)c3)c3ccccc23)c1] | A6 |
| 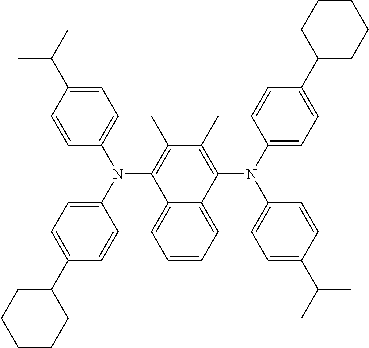 [SMILES: Cc1c(C)c(N(c2ccc(C(C)C)cc2)c2ccc(C3CCCCC3)cc2)c2ccccc2c1N(c1ccc(C(C)C)cc1)c1ccc(C2CCCCC2)cc1] | A7 |
| 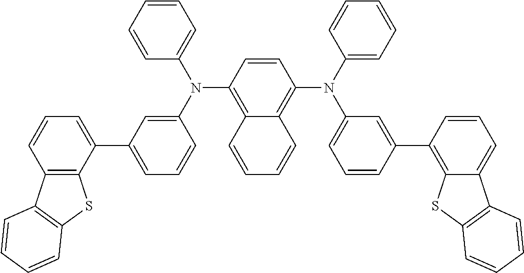 [SMILES: c1ccc(N(c2cccc(-c3cccc4c3sc3ccccc34)c2)c2ccc(N(c3ccccc3)c3cccc(-c4cccc5c4sc4ccccc45)c3)c3ccccc23)cc1] | A8 |
| 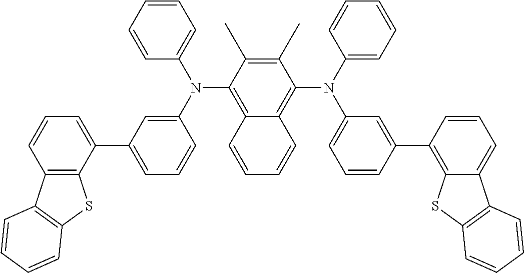 [SMILES: Cc1c(C)c(N(c2ccccc2)c2cccc(-c3cccc4c3sc3ccccc34)c2)c2ccccc2c1N(c1ccccc1)c1cccc(-c2cccc3c2sc2ccccc23)c1] | A9 |
| 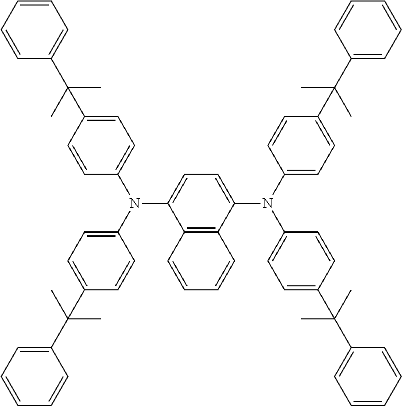 [SMILES: CC(C)(c1ccccc1)c1ccc(N(c2ccc(C(C)(C)c3ccccc3)cc2)c2ccc(N(c3ccc(C(C)(C)c4ccccc4)cc3)c3ccc(C(C)(C)c4ccccc4)cc3)c3ccccc23)cc1] | A10 |
| 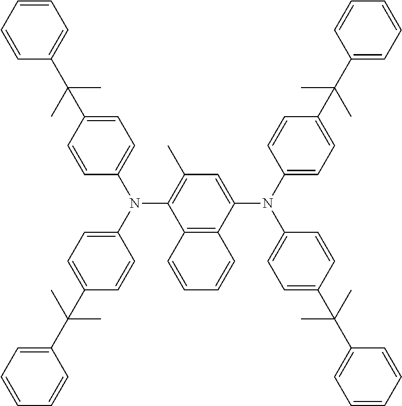 [SMILES: Cc1cc(N(c2ccc(C(C)(C)c3ccccc3)cc2)c2ccc(C(C)(C)c3ccccc3)cc2)c2ccccc2c1N(c1ccc(C(C)(C)c2ccccc2)cc1)c1ccc(C(C)(C)c2ccccc2)cc1] | A11 |
| 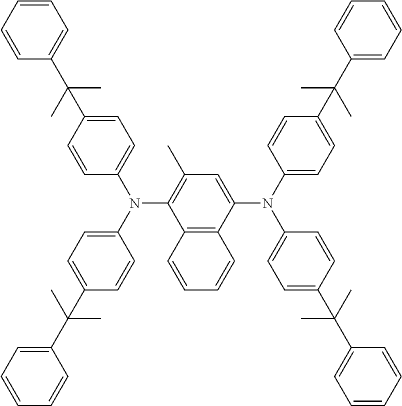 [SMILES: Cc1cc(N(c2ccc(C(C)(C)c3ccccc3)cc2)c2ccc(C(C)(C)c3ccccc3)cc2)c2ccccc2c1N(c1ccc(C(C)(C)c2ccccc2)cc1)c1ccc(C(C)(C)c2ccccc2)cc1] | A12 |
| 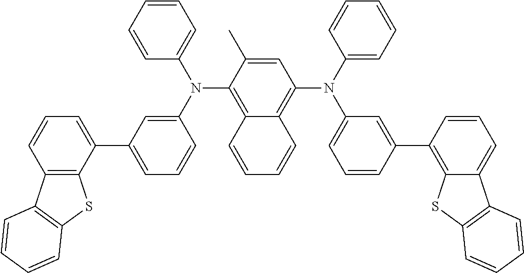 [SMILES: Cc1cc(N(c2ccccc2)c2cccc(-c3cccc4c3sc3ccccc34)c2)c2ccccc2c1N(c1ccccc1)c1cccc(-c2cccc3c2sc2ccccc23)c1] | A13 |

The compound of formula (I) can be synthesized based on the following reaction scheme 1, but it is not limited thereto.

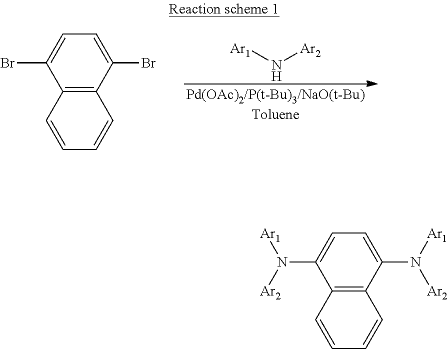

The present disclosure further provides an organic electroluminescent element, comprising: a cathode; an anode; and an organic layer comprising a compound of formula (I) of the present disclosure and formed between the cathode and the anode.

The compound of formula (I) can be used in the organic layer of an OLED. Therefore, the OLED of the present disclosure has at least one organic layer disposed between the anode and cathode on a substrate, wherein the organic layer comprises the compound of formula (I) above. The organic layer can be a light emitting layer, a hole blocking layer, an electron transport layer, an electron injection layer, or a hole transport layer. In addition to the organic layer, the organic electroluminescent element can further include at least one layer, which differs from the organic layer and is selected from an electron transport layer, an electron injection layer, a light emitting layer, a hole blocking layer, and an electron blocking layer.

In one embodiment, the organic layer comprising the compound of formula (I) is a light emitting layer, which comprises a fluorescent material or a phosphorescent material. Thus, the OLED of the present disclosure emits fluorescent or phosphorescent light.

In one embodiment, the light emitting layer comprises the compound of formula (I) as a guest emitter and a fluorescent material as a host emitter. Thus, the OLED of the present disclosure emits fluorescent light.

In one embodiment, the content of the compound of formula (I) is 2 wt %, 3 wt %, 4 wt % to 13 wt %, 14 wt %, and 15 wt %, based on weight of the light emitting layer.

In one embodiment, the fluorescent material has an HOMO energy level of 5.7 eV to 5.9 eV (preferably 5.8 V), and has an LUMO energy level of 2.6 eV to 2.8 eV (preferably 2.7 eV).

In one embodiment, the content of the compound of formula (I) is 25 wt % to 100 wt %, based on weight of the organic layer. Also, the organic layer has a thickness of 1 nm to 500 nm.

In one embodiment, a hole injection layer and a hole transport layer are further comprised between the anode and the organic layer, and an electron transport layer and an electron injection layer are further comprised between the organic layer and the cathode, wherein the organic layer is a light emitting layer.

The structure of the organic electroluminescent element of the present disclosure will be illustrated with reference to the figures.

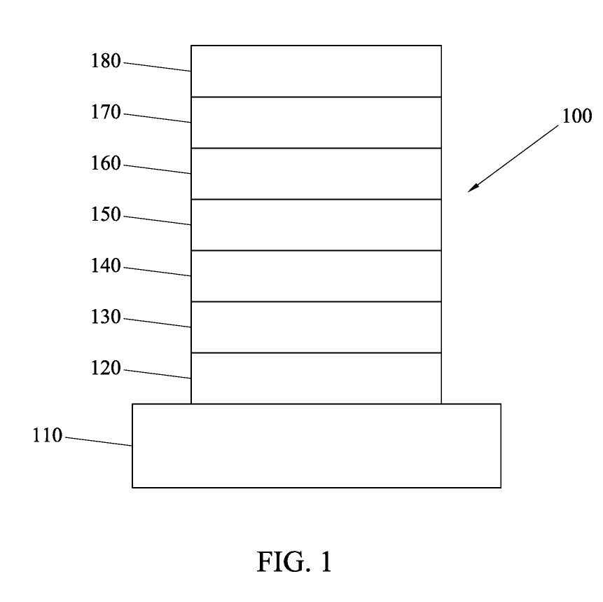

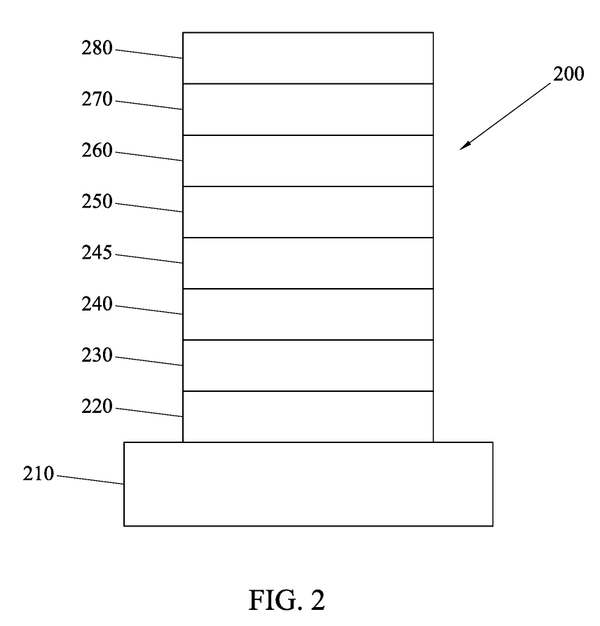

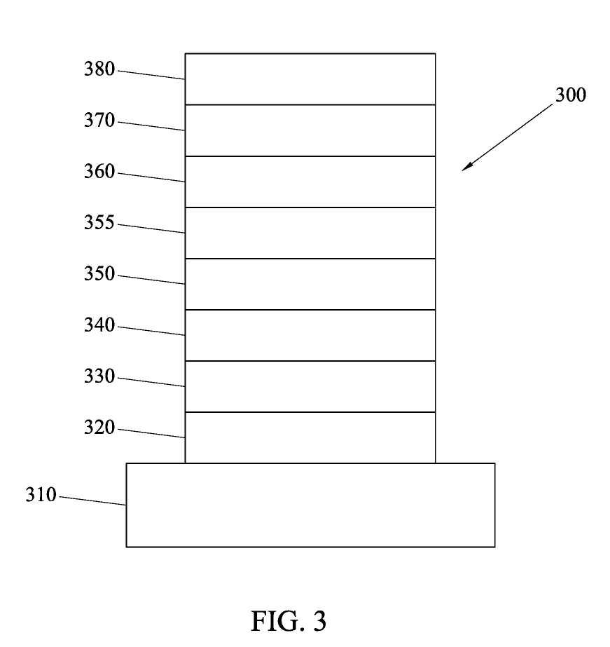

FIG. 1 is a schematic sectional view of an organic electroluminescent element of one example of the present disclosure. The organic electroluminescent element 100 comprises a substrate 110, an anode 120, a hole injection layer 130, a hole transport layer 140, a light emitting layer 150, an electron transport layer 160, an electron injection layer 170, and a cathode 180. The organic electroluminescent element 100 can be prepared by depositing above layers sequentially. FIG. 2 is a schematic sectional view of an organic electroluminescent element of another example of the present disclosure. The organic electroluminescent element shown in FIG. 2 is similar to that shown in FIG. 1 by comprising a substrate 210, an anode 220, a hole injection layer 230, a hole transport layer 240, a light emitting layer 250, an electron transport layer 260, an electron injection layer 270, and a cathode 280, except that an exciton blocking layer 245 is disposed between the hole transport layer 240 and the light emitting layer 250. FIG. 3 shows a schematic sectional view of the organic electroluminescent element of still another example of the present disclosure. The organic electroluminescent element shown in FIG. 3 is also similar to that shown in FIG. 1 by comprising a substrate 310, an anode 320, a hole injection layer 330, a hole transport layer 340, a light emitting layer 350, an electron transport layer 360, an electron injection layer 370, and a cathode 380, except that an exciton blocking layer 355 is disposed between the light emitting layer 350 and the electron transport layer 360.

An organic electroluminescent element can also be manufactured according to reverse structures of the elements shown in FIGS. 1-3. In the reverse structures, one or more layer(s) can be increased or decreased optionally.

Materials applied in a hole injection layer, a hole transport layer, an electron blocking layer, a hole blocking layer, a light emitting layer, and an electron injection layer can be selected from those commonly used. For example, an electron transport material forming an electron transport layer differs from a material forming a light emitting layer, and has the property for transporting holes, such that it facilitates migration of holes in the electron transport layer and prevents carrier accumulation due to dissociation energy gap between the light emitting layer and the electron transport layer.

In addition, U.S. Pat. No. 5,844,363 describes a flexible transparent substrate bound with an anode, the entire content of which is cited herein. The p-doped hole transport layer illustrated in US 20030230980A1 is m-MTDATA doped with F4-TCNQ at a molar ratio of 50:1, the entire content of US 20030230980A1 is cited herein. The n-doped electron transport layer illustrated in US 20030230980A1 is BPhen doped with lithium at a molar ratio of 1:1. The entire contents of the cathodes illustrated in U.S. Pat. No. 5,703,436 and U.S. Pat. No. 5,707,745 are cited herein, the cathode has a metal thin layer such as Mg/Ag (Mg:Ag) and a transparent electro-conductive layer (ITO Layer) covered with a metal layer which is formed by sputtering. U.S. Pat. No. 6,097,147 and US 20030230980A1 describe applications and principles of various blocking layers, the entire contents of which are cited herein. US 20040174116 A1 illustrates injection layers and protective layers, the entire content of which is incorporated herein.

The structures and materials without specific descriptions can also be used in the present disclosure. For example, U.S. Pat. No. 5,247,190 describes an organic electroluminescent element including a polymer material (PLEDs), the entire content of which is incorporated cited herein. Furthermore, the entire content of an organic electroluminescent element having a single organic layer or an organic electroluminescent element formed by stacking, as described in U.S. Pat. No. 5,707,745, is incorporated herein.

Unless restricted particularly or limited otherwise, any layers in different examples can be formed by any suitable deposition methods. For an organic layer, the preferred method includes, for example, thermal evaporation and jet printing described in U.S. Pat. No. 6,013,982 and U.S. Pat. No. 6,087,196, the entire contents of which are incorporated cited herein; the organic vapor phase deposition (OVPD) described in US 6337102, the entire content of which is cited incorporated herein; US 20080233287A1 describes deposition by organic vapor jet printing (OVJP) described in US 20080233287A1, the entire content of which is incorporated herein. Other suitable methods include spin coating and solution-based processes. The solution-based processes are carried out in a nitrogen or inert gas atmosphere preferably. For other layers, the preferred method includes thermal evaporation. The preferred patterning methods includes, for example, the process of deposition through mask, following by cold welding and the process of integration of jet printing or organic vapor jet printing and patterning described in U.S. Pat. No. 6,294,398 and U.S. Pat. No. 6,468,819, the entire contents of which are incorporated herein. Certainly, other methods can also be used. The materials used in deposition can be adjusted based on the particular deposition method used.

An amorphous thin film used in an organic electroluminescent element can be prepared from the compound of formula (I) of the present disclosure by vacuum deposition or a spin coating method.

When the compound of formula (I) is used in a light emitting layer, the organic electroluminescent element emits light of different blue colors (such as pale blue, blue, or dark blue) with high color purity.

The organic electroluminescent element of the present disclosure can be applied in a single element having the structure of array arrangement or an element having a cathode and an anode arranged in the X-Y coordinate of an array. In addition, when used in a full color or multiple color display device, the organic electroluminescent element of the present disclosure can achieve better performance and emit white light.

Hereinafter, various properties and effects of the present disclosure are explained in detail by the following examples. The detailed examples are used for explaining properties of the present disclosure only, and the present disclosure is not limited to those illustrated in specific examples.

### SYNTHETIC EXAMPLE 1

### Compound A1

1,4-dibromonaphthalene (5 g, 17.48 mmol) and N-([1,1′-biphenyl]-4-yl)-9,9-dimethyl-9H-fluoren-2-amine (13.9 g, 38.64 mmol) were 5 added in a reaction flask. After the toluene was sufficiently dry, dry toluene (50 mL) was added thereto, and then the mixture was heated and stirred. Sodium t-butoxide (4.36 g, 45.4 mmol) was added when the mixture was heated to 50° C., and the mixture was heated and stirred again. Pd(OAc)₂ (0.117 g, 0.525 mmol) and tri-tert-butylphosphine (0.302 g, 1.399 mmol) were added when the temperature reached 60° C., and the reaction underwent overnight. After the reaction completed, deionized water (300 mL) was added, and the mixture was stirred for 30 minutes. Solid was filtered, and the organic layer was collected after removing the aqueous layer from the filtrate. The organic layer was purified through silicon chromatography, and the purified organic layer and the solid filtered were combined and concentrated via distillation. Methanol (350 mL) was added for precipitation, and the precipitate was filtered to obtain Compound A1 (6.9 g) as a pale-yellow solid.

¹H NMR (400 MHz, CDCl₃, δ):

δ 8.105-8.05 (dd, 3H); 7.67-7.63 (d, 3H); 7.63-7.57 (d, 3H); 7.525-7.475 (d, 6H); 7.45-7.225 (m, 27H); 7.2-7.1375 (d, 6H); 7.1-7.05 (m, 2H).

### SYNTHETIC EXAMPLE 2

### Compound A2

1,4-dibromonaphthalene (⁷ g, 24.48 mmol) and 4-cyclohexyl-N-(4-isopropylphenyl)aniline (15.08 g, 51.4 mmol) were added in a reaction flask. After the toulene was sufficiently dry, dry toluene (50 mL) was added thereto, and then the mixture was heated and stirred. Sodium t-butoxide (6.11 g, 63.6 mmol) was added when the mixture was heated to 50° C., and the mixture was heated and stirred again. Pd(OAc)₂ (0.165 g, 0.734 mmol) and tri-tert-butylphosphine (0.423 g, 1.958 mmol) were added when the temperature reached 60° C., and the reaction underwent overnight. After the reaction completed, deionized water (300 mL) was added, and the mixture was stirred for 30 minutes. Solid was filtered, and the organic layer collected after removing the aqueous layer from the filtrate. The organic layer was purified through silicon chromatography, and the purified organic layer and the solid filtered were combined and concentrated via distillation. Methanol (350 mL) was added for precipitation, and the precipitate was filtered to obtain Compound A2 (15.5 g) as a pale-yellow solid.

¹H NMR (400 MHz, CDCl₃, δ):

δ 8.04-7.985 (dd, 5H); 7.345-7.275 (m, 10H); 7.086-7.028 (m, 23H); 7.028-6.958 (m, 20H).

### SYNTHETIC EXAMPLE 3

### Compound A3

1,4-dibromonaphthalene (2 g, 6.99 mmol) and di-m-tolylamine (2.89 g, 14.687 mmol) were added in a reaction flask. After the toluene was sufficiently dry, dry toluene (30 mL) was added thereto, and then the mixture was heated and stirred. Sodium t-butoxide (1.74 g, 18.18 mmol) was added when the mixture was heated to 50° C., and the mixture was heated and stirred again. Pd(OAc)₂ (0.047 g, 0.21 mmol) and tri-tert-butylphosphine (0.12 g, 0.56 mmol) were added when the temperature reached 60° C., and the reaction underwent overnight. After the reaction completed, deionized water (50 mL) was added, and the mixture was stirred for 30 minutes. Solid was filtered, and the organic layer was collected after removing the aqueous layer from the filtrate. The organic layer was purified through silicon chromatography, and the purified organic layer and the solid filtered were combined and concentrated via distillation. Methanol (100 mL) was added for precipitation, and the precipitate was filtered to obtain Compound A3 (3 g) as a pale-yellow solid.

¹H NMR (400 MHz, CDCl₃, δ):

δ 8.03-7.965 (dd, 3H); 7.345-7.24 (m, 7H); 7.12-7.06 (t, 6H); 6.91-6.86 (s, 6H); 6.85-6.8 (d, 6H); 6.85-6.74 (d, 6H).

### SYNTHETIC EXAMPLE 4

### Compound A4

1,4-Dibromo-2-methylnaphthalene (3 g, 10 mmol) and di-m-tolylamine (4.14 g, 21 mmol) were added in a reaction flask. After the toluene was sufficiently dry, dry toluene (35 mL) was added thereto, and then the mixture was heated and stirred. Sodium t-butoxide (2.49 g, 2.6 mmol) was added when the mixture was heated to 50° C., and the mixture was heated and stirred again. Pd(OAc)₂ (0.067 g, 0.3 mmol) and tri-tert-butylphosphine (0.1728 g, 0.8 mmol) were added when the temperature reached 60° C., and the reaction underwent overnight. After the reaction completed, deionized water (50 mL) was added, and the mixture was stirred for 30 minutes. Solid was filtered, and the organic layer was collected after removing the aqueous layer from the filtrate. The organic layer was purified through silicon chromatography, and the purified organic layer and the solid filtered were combined and concentrated via distillation. Methanol (100 mL) was added for precipitation, and the precipitate was filtered to obtain Compound A4 (1.8 g) as a pale-yellow solid.

¹H NMR (400 MHz, CDCl₃, δ):

δ 7.97-7.9 (dd, 3H); 7.35-7.24 (m, 5H); 7.23-7.1 (s, 2H); 7.14-7.05 (m, 8H); 6.91-6.86 (s, 6H); 6.83-6.8 (t, 6H); 6.79-6.745 (d, 3H); 6.74-6.685 (d, 3H).

### SYNTHETIC EXAMPLE 5

### Compound A5

1,4-dibromo-2-methylnaphthalene (3 g, 10 mmol) and 4-cyclohexyl-N-(4-isopropylphenyl)aniline) (6.456 g, 22 mmol) were added in a reaction flask. After the toluene was sufficiently dry, dry toluene (35 mL) was added thereto, and then the mixture was heated and stirred. Sodium t-butoxide (2.49 g, 2.6 mmol) was added when the mixture was heated to 50° C., and the mixture was heated and stirred again. Pd(OAc)₂ (0.067 g, 0.3 mmol) and tri-tert-butylphosphine (0.1728 g, 0.8 mmol) was added when the temperature reached 60° C., and the reaction underwent overnight. After the reaction completed, deionized water (50 mL) was added, and the mixture was stirred for 30 minutes. Solid was filtered, and the organic layer collected after removing the aqueous layer from the filtrate. The organic layer was purified through silicon chromatography, and the purified organic layer and the solid filtered were combined and concentrated via distillation. Methanol (100 mL) was added for precipitation, and the precipitate was filtered to obtain Compound A5 (12 g) as a pale-yellow solid.

¹H NMR (400 MHz, CDCl₃, δ):

δ 7.965-7.885 (dd, 4H); 7.325-7.25 (m, 15H); 7.1-6.91 (m, 41H).

### SYNTHETIC EXAMPLE 6

### Compound A8

1,4-dibromonaphthalene (2 g, 6.99 mmol) and 3-(dibenzo[b,d]thiophen-4-yl)-N-phenylaniline (5.16 g, 14.7 mmol) were added in a reaction flask. After the toluene was sufficiently dry, dry toluene (25 mL) was added thereto, and then the mixture was heated and stirred. Sodium t-butoxide (1.74 g, 17.18 mmol) was added when the mixture was heated to 50° C., and the mixture was heated and stirred again. Pd(OAc)₂ (0.047 g, 0.21 mmol) and tri-tert-butylphosphine (0.12 g, 0.56 mmol) were added when the temperature reached 60° C., and the reaction underwent overnight. After the reaction completed, deionized water (50 mL) was added, and the mixture was stirred for 30 minutes. Solid was filtered, and the organic layer collected after removing the aqueous layer from the filtrate. The organic layer was purified through silicon chromatography, and the purified organic layer and the solid filtered were combined and concentrated via distillation. Methanol (100 mL) was added for precipitation, and the precipitate was filtered to obtain Compound A8 (2.5 g) as a pale-yellow solid.

¹H NMR (400 MHz, CDCl₃, δ):

δ 8.22-8.12 (S, 3H); 8.1-7.94 (m, 5H); 7.62-6.82 (m, 30).

### SYNTHETIC EXAMPLE 7

### Compound A9

1,4-dibromo-2, 3-dimethylnaphthalene (2 g, 6.37 mmol) and 3-(dibenzo[b,d]thiophen-4-yl)-N-phenylaniline (2 g, 6.37 mmol) were added in a reaction flask. After the toluene was sufficiently dry, dry toluene (25 mL) was added thereto, and then the mixture was heated and stirred. Sodium t-butoxide (1.589 g, 16.6 mmol) was added when the mixture was heated to 50° C., and the mixture was heated and stirred again. Pd(OAc)₂ (0.0428 g, 0.191 mmol) and tri-tert-butylphosphine (0.11 g, 0.55 mmol) were added when the temperature reached 60° C., and the reaction underwent overnight. After the reaction completed, deionized water (50 mL) was added, and the mixture was stirred for 30 minutes. Solid was filtered, and the organic layer was collected after removing the aqueous layer from the filtrate. The organic layer was purified through silicon chromatography, and the purified organic layer and the solid filtered were combined and concentrated via distillation. Methanol (100 mL) was added for precipitation, and the precipitate was filtered to obtain Compound A9 (1.8 g) as a pale-yellow solid.

¹H NMR (400 MHz, CDCl₃, δ):

δ 8.14-8.12 (d, 2H); 8.19-8.04 (m, 4H); 7.99-7.92 (m, 3H); 7.62-7.3 (m, 17H); 7.26-7.25 (s, 1H); 7.22-7.08 (m, 7H); 7.1-6.91 (m, 4H); 6.79-6.7 (m, 4H).

### SYNTHETIC EXAMPLE 8

### Compound A10

1,4-dibromonaphthalene (5 g, 17.48 mmol) and bis(4-(2-phenylpropan-2-yl)phenyl)amine (14.9 g, 36.71 mmol) were added in a reaction flask. After the toluene was sufficiently dry, dry toluene (50 mL) was added thereto, and then the mixture was heated and stirred. Sodium t-butoxide (4.36 g, 45.4 mmol) was added when the mixture was heated to 50° C., and the mixture was heated and stirred again. Pd(OAc)₂ (0.117 g, 0.525 mmol) and tri-tert-butylphosphine (0.302 g, 1.399 mmol) were added when the temperature reached 60° C., and the reaction underwent overnight. After the reaction completed, deionized water (300 mL) was added, and the mixture was stirred for 30 minutes. Solid was filtered, and the organic layer was collected after removing the aqueous layer from the filtrate. The organic layer was purified through silicon chromatography, and the purified organic layer and the solid filtered were combined and concentrated via distillation Methanol (350 mL) was added for precipitation, and the precipitate was filtered to obtain Compound A10 (8.5 g) as a pale-green solid.

¹H NMR (400 MHz, CDCl₃, δ):

δ 8.0-7.92 (m, 3H); 7.33-7.23 (m, 32H); 7.2-7.12 (m, 6H); 7.07-7.00 (d, 13H);

6.95-6.88 (d, 12H).

### EXAMPLE 1

### Manufacture of an Organic Luminescent Element Emitting Blue Light

A substrate was washed with a solvent and ozone under UV light for degreasing, prior to loading the substrate in an evaporation system for use. Thereafter, the substrate was transferred in a vacuum deposition chamber, and all of the layers were deposited on the top of the substrate. The layers shown in FIG. 2 were deposited sequentially at a vacuum of about 10⁻⁶ Torr using a heated evaporation boat as following:
a) an indium tin oxide (ITO) layer;
b) a hole injection layer with a thickness of 20 nm, HAT-CN;
c) a hole transport layer with a thickness of 60 nm, HT;
d) a light emitting layer with a thickness of 30 nm, containing 3 wt % of Compound A2 as a guest emitter and BH1 as a host emitter (BH1 is a product name of E-ray Optoelectronics Technology Co., Ltd.);
e) an electron transport layer with a thickness of 25 nm, containing ET and doped lithium quinolinate (Liq);
f) an electron injection layer with a thickness of 1 nm, lithium fluoride (LiF); and
g) a cathode with a thickness of 150 nm, containing aluminum (Al).

The structure of the element of Example 1 can be represented as: ITO/HAT-CN (20 nm)/HT (60 nm)/BH1: 3 wt % of Compound A2 (30 nm)/50 wt % of ET: 50 wt % of Liq (25 nm)/LiF (1 nm)/Al (150 nm).

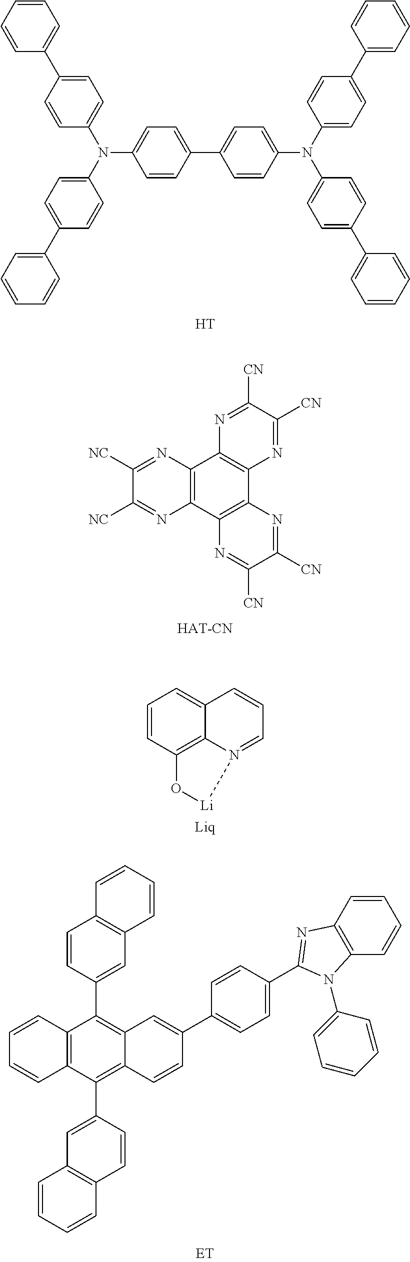

After above layers were deposited, the element was transferred from the deposition chamber to a drying oven and then encapsulated with an UV-curable epoxy resin and a glass cover-plate containing a moisture absorbent. The organic electroluminescent element has a light emitting area of 9 mm².

The electroluminescent properties of the organic electroluminescent element thus manufactured was determined at room temperature using a constant current source (KEITHLEY 2400 Source Meter, made by Keithley Instruments, Inc., Cleveland, Ohio) and a photometer (PHOTO RESEARCH SpectraScan PR 650, made by Photo Research, Inc., Chatsworth, Calif.).

The measurements were performed by driving the constant current according to light color at room temperature and different initial luminance. The light color was represented by coordinates set forth by the International Commission on Illumination.

### EXAMPLE 2

### Manufacture of an Organic Electroluminescent Element

Except using Compound A5 as the guest emitter instead of Compound A2 in the light emitting layer of Example 1, the others were the same as those in the structure in Example 1.

The structure of the element of Example 2 can be represented as: ITO/HAT-CN (20 nm)/HT (60 nm)/BH1: 3 wt % of Compound A5 (30 nm)/50 wt % of ET: 50 wt % Liq (25 nm)/LiF (1 nm)/Al (150 nm).

### EXAMPLE 3

### Manufacture of an Organic Electroluminescent Element

Except using Compound A6 as the guest emitter instead of Compound A2 in the light emitting layer of Example 1, the others were the same as those in the structure in Example 1.

The structure of the element of Example 3 can be represented as: ITO/HAT-CN (20 nm)/HT (60 nm)/BH1: 3 wt % of Compound A6 (30 nm)/50 wt % of ET: 50 wt % of Liq (25 nm)/LiF (1 nm)/Al (150 nm).

### EXAMPLE 4

### Manufacture of an Organic Electroluminescent Element

Except using Compound A8 as the guest emitter instead of Compound A2 in the light emitting layer of Example 1, the others were the same as those in the structure in Example 1.

The structure of the element of Example 4 can be represented as: ITO/HAT-CN (20 nm)/HT (60 nm)/BH1: 3 wt % of Compound A8 (30 nm)/50 wt % of ET: 50 wt % of Liq (25 nm)/LiF (1 nm)/A1 (150 nm).

### EXAMPLE 5

### Manufacture of an Organic Electroluminescent Element

Except using Compound A10 as the guest emitter instead of Compound A2 in the light emitting layer of Example 1, the others were the same as those in the structure in Example 1.

The structure of the element of Example 5 can be represented as: ITO/HAT-CN (20 nm)/HT (60 nm)/BH1: 3 wt % of Compound A10 (30 nm)/50 wt % of ET: 50 wt % of Liq (25 nm)/LiF (1 nm)/A1 (150 nm).

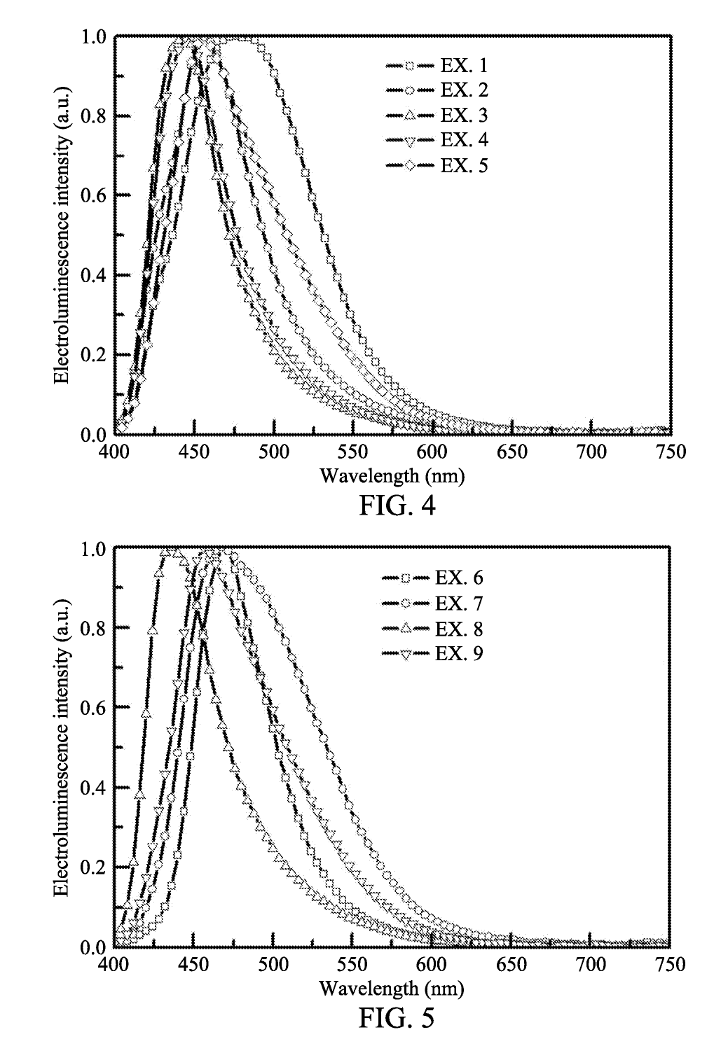

The organic electroluminescent elements manufactured in Examples 1-5 using BH1 as the host emitter material had emission peaks, the maximum luminous efficiencies, diving voltages, power efficiencies and CIE coordinates listed in Table 2. The electroluminescent spectra of the organic electroluminescent elements manufactured in Examples 1-5 are shown in FIG. 4.

TABLE 2

| | | | | Maximum | | |
|---|---|---|---|---|---|---|
| | | | | luminescent | Power | |
| | | Emitting | Driving | efficiency | efficiency | |
| | Compound in light | peak | voltage | (cd/A) @ | (lm/W) | |
| | emitting layer | (nm) | (V) | 10 mA/cm² | 10 mA/cm² | CIE(x, y) |
| Ex. 1 | Compound A2 | 476 | 4.37 | 8.01 | 5.76 | (0.162, 0.254) |
| Ex. 2 | Compound A5 | 456 | 4.43 | 3.51 | 2.49 | (0.155, 0.134) |
| Ex. 3 | Compound A6 | 444 | 4.66 | 2.22 | 1.50 | (0.159, 0.087) |
| Ex. 4 | Compound A8 | 448 | 4.41 | 3.17 | 2.26 | (0.156, 0.099) |
| Ex. 5 | Compound A10 | 456 | 4.51 | 5.76 | 4.01 | (0.160, 0.164) |

### EXAMPLE 6

### Manufacture of an Organic Electroluminescent Element

Except using Compound A1 instead of Compound A2 in the light emitting layer of Example 1 as the guest emitter and using BH2 (BH2 is a product name of E-ray Optoelectronics Technology Co., Ltd.) instead of BH1 as the host emitter, the others were the same as those in the structure in Example 1.

The structure of the element of Example 6 can be represented as: ITO/HAT-CN (20 nm)/HT (60 nm)/BH2: 3 wt % of Compound A1 (30 nm)/50 wt % of ET: 50 wt % of Liq (25 nm) LiF (1 nm)/Al (150 nm).

### EXAMPLE 7

### Manufacture of an Organic Electroluminescent Element

Except using BH2 as the host emitter instead of BH1 in the light emitting layer of Example 2, the others were the same as those in the structure in Example 1.

The structure of the element of Example 7 can be represented as: ITO/HAT-CN (20 nm)/HT (60 nm)/BH2: 3 wt % of Compound A2 (30 nm)/50 wt % of ET: 50 wt % of Liq (25 nm)/LiF (1 nm)/Al (150 nm).

### EXAMPLE 8

### Manufacture of an Organic Electroluminescent Element

Except using Compound A4 instead of Compound A2 in the light emitting layer of Example 1 as the guest emitter and using BH2 instead of BH1 as the host emitter, the others were the same as those in the structure in Example 1.

The structure of the element of Example 8 can be represented as: ITO/HAT-CN (20 nm)/HT (60 nm)/BH2: 3 wt % of Compound A4 (30 nm)/50 wt % of ET: 50 wt % of Liq (25 nm)/LiF (1 nm)/A1 (150 nm).

### EXAMPLE 9

### Manufacture of an Organic Electroluminescent Element

Except using Compound A10 instead of Compound A2 in the light emitting layer of Example 1 as the guest emitter and using BH2 instead of BH1 as the host emitter, the others were the same as those in the structure in Example 1.

The structure of the element of Example 9 can be represented as: ITO/HAT-CN (20 nm)/HT (60 nm)/BH2: 3 wt % of Compound A10 (30 nm)/50 wt % of ET: 50 wt % of Liq (25 nm)/LiF (1 nm)/A1 (150 nm).

The organic electroluminescent elements manufactured in Examples 6-9 using BH2 as the host material had emission peaks, the maximum luminous efficiencies, diving voltages, power efficiencies and CIE coordinates listed in Table 3. The electroluminescent spectra of the organic electroluminescent elements manufactured in Examples 6-9 are shown in FIG. 5.

TABLE 3

| | | | | The maximum | | |
|---|---|---|---|---|---|---|
| | | | | luminescent | Power | |
| | | Emitting | Driving | efficiency | efficiency | |
| | Compound in the light | peak | voltage | (cd/A) @ | (lm/W) | |
| | emitting layer | (nm) | (V) | 10 mA/cm² | 10 mA/cm² | CIE(x, y) |
| Ex. 6 | compound A1 | 468 | 4.26 | 4.45 | 3.28 | (0.142, 0.191) |
| Ex. 7 | compound A2 | 468 | 4.52 | 6.43 | 4.47 | (0.173, 0.274) |
| Ex. 8 | compound A4 | 436 | 4.46 | 2.21 | 1.56 | (0.167, 0.099) |
| Ex. 9 | compound A10 | 456 | 4.52 | 5.77 | 4.00 | (0.161, 0.196) |

The present disclosure is not limited to the Examples, methods and embodiments above, and is based on all of the examples and methods fallen within the claims and the spirit of the present disclosure.

As discussed above, the organic electroluminescent element of the present disclosure using the diamine compound of formula (I) as a guest emitter can emit light with different blue colors, such as pale blue (Compounds A1 and A2), blue (Compounds A5 and A10) and dark blue (Compounds A4, A6, and A8), such that it can be used widely in OLED displays and light sources. Therefore, the organic electroluminescent element of the present disclosure have very high technical values, such that it is suitable for use in flat panel displays, displays of mobile communication elements, OLED light sources, and sign-boards.

1. A diamine compound represented by formula (I): 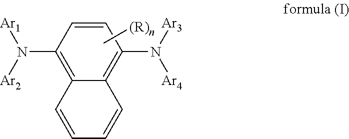
wherein
R represents substituted or unsubstituted (C1-C10)alkyl;
each of Ar₁, Ar₂, Ar₃ and Ar₄ independently represents substituted or unsubstituted (C6-C30)aryl, or substituted or unsubstituted (5 to 30-membered)heteroaryl; and
n represents an integer of from 0 to 4.

2. The diamine compound of claim 1, wherein Ar₁ and Ar₃ are the same, and Ar₂ and Ar₄ are the same.

3. The diamine compound of claim 1, wherein
R represents (C1-C10)alkyl;
each of Ar₁, Ar₂, Ar₃ and Ar₄ independently represents substituted (C6-C30)aryl, wherein the substituent of the substituted (C6-C30)aryl is at least one selected from the group consisting of (C1-C10)alkyl, (C1-C10)alkyl substituted with (C6-C30)aryl, (C3-C10)cycloalkyl, and (5 to 30-membered)heteroaryl; and
n represents 0, 1, or 2.

4. The diamine compound of claim 1, wherein at least one of Ar₁, Ar₂, Ar₃ and Ar₄ is (C6-C30)aryl substituted with (C1-C10)alkyl or (C3-C10)cycloalkyl.

5. The diamine compound of claim 1, wherein at least one of Ar₁, Ar₂, Ar₃ and Ar₄ is (C6-C30)aryl substituted with (C2-C10)alkyl or (C3-C10)cycloalkyl.

6. The diamine compound of claim 1, wherein n is 0, and at least one of Ar₁, Ar₂, Ar₃ and Ar₄ is (C6-C30)aryl substituted with (C3-C10)alkyl or (C3-C10)cycloalkyl.

7. The diamine compound of claim 1, wherein n is 0, and each of Ar₁, Ar₂, Ar₃ and Ar₄ is (C6-C30)aryl substituted with (C3-C10)alkyl or (C3-C10)cycloalkyl.

8. The diamine compound of claim 1, wherein each of Ar₁, Ar₂, Ar₃ and Ar₄ independently represents 9,9-dimethylfluorenyl, biphenyl, propylphenyl, cyclohexylphenyl, methylphenyl, phenyl, dibenzothienylphenyl, or phenylpropylphenyl.

9. The diamine compound of claim 1, which is represented by formula (II): 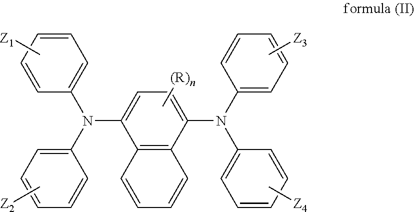
wherein
R represents substituted or unsubstituted (C1-C10)alkyl;
each of Z₁, Z₂, Z₃ and Z₄ independently represents substituted or unsubstituted (C1-C10)alkyl, substituted or unsubstituted (C3-C10)cycloalkyl, substituted or unsubstituted (C6-C30)aryl, or substituted or unsubstituted (5 to 30-membered)heteroaryl; or at least one of Z₁, Z₂, Z₃ and Z₄ is fused with an adjacent benzene ring of formula (II) to form substituted or unsubstituted (C9-C30)aryl; or at least one of Z₁, Z₂, Z₃ and Z₄ is fused with the adjacent benzene ring of formula (II) to form substituted or unsubstituted (10 to 30-membered)heteroaryl; and
n represents an integer of from 0 to 4.

10. The diamine compound of claim 9, wherein at least one of Z₁, Z₂, Z₃ and Z₄ is substituted with at least one substituent selected from the group consisting of (C—C10)alkyl, (C3-C10)cycloalkyl, (C6-C30)aryl, and (5 to 30-membered)heteroaryl.

11. The diamine compound of claim 9, wherein
R represents (C1-C10)alkyl;
each of Z₁, Z₂, Z₃ and Z₄ independently represents methyl, isopropyl, phenylisopropyl, cyclohexyl, or dibenzothienyl; or at least one of Z₁, Z₂, Z₃ and Z₄ is fused with the adjacent benzene ring of formula (II) to form 9,9-dimethylfluorenyl; and
n represents 0, 1, or 2.

12. An organic electroluminescent element, comprising:
a cathode;
an anode; and
an organic layer comprising the diamine compound of claim 1 and formed between the cathode and the anode.

13. The organic electroluminescent element of claim 12, wherein the diamine compound is in an amount of from 25 wt % to 100 wt %, based on a weight of the organic layer, and the organic layer has a thickness of from 1 nm to 500 nm.

14. The organic electroluminescent element of claim 12, wherein the organic layer is an electron transport layer, an electron injection layer, a light emitting layer, a hole blocking layer, or an electron blocking layer.

15. The organic electroluminescent element of claim 12, wherein the organic layer is a light emitting layer.

16. The organic electroluminescent element of claim 15, wherein the diamine compound is in an amount of from 2 wt % to 15 wt %, based on a weight of the light emitting layer.

17. The organic electroluminescent element of claim 15, wherein the diamine compound in the light emitting layer serves as a guest emitter and the light emitting layer further comprises a fluorescent material serving as a host emitter.

18. The organic electroluminescent element of claim 17, wherein the fluorescent material has an HOMO energy level of from 5.7 eV to 5.9 eV.

19. The organic electroluminescent element of claim 12, which emits blue light.

20. An organic electroluminescent device comprising the organic electroluminescent element of claim 12 and emitting white light.
